(19) United States
(12) Patent Application Publication
(10) Pub. No.: US 2026/0123840 A1
(43) Pub. Date: May 7, 2026

(54) VITAL SIGNS MEASURING DEVICE

(71) Applicant: BODYLOG CORPORATION, Seoul (KR)

(72) Inventors: In Ho KIM, Seoul (KR); Jong Hun KIM, Seoul (KR)

(73) Assignee: BODYLOG CORPORATION, Seoul (KR)

(21) Appl. No.: 19/102,807

(22) PCT Filed: Aug. 9, 2023
(86) PCT No.: PCT/KR2023/011795

§ 371 Date: Jan. 20, 2026

(30) Foreign Application Priority Data
Aug. 10, 2022 (KR) 1020220100186

(51) Int. Cl. A61B 5/0205; A61B 5/00

(52) U.S. Cl. CPC A61B 5/0205; A61B 5/0002; A61B 5/4561; A61B 2560/0214; A61B 2560/0223; A61B 2562/0219

(57) ABSTRACT

A vital signs measuring device includes a posture measuring unit attachable within a pentahedral space of a body, a biosignal measuring unit detachably connected to the posture measuring unit and capable of measuring at least one of the body's vital signs, a wireless communication unit that transmits data calculated from the biosignal measuring unit externally when the posture measuring unit determines the body to be in a specific posture, and a power supply unit that supplies power to the posture measuring unit, biosignal measuring unit and wireless communication unit. This enables vital signs data to be obtained that is stable and reliable for specific body postures.

### TECHNICAL FIELD

Example embodiments of the present invention relate to a vital signs measuring device configured to measue body vital signs including a posture determination unit. More particularly, example embodiments of the present invention relate to a vital signs measuring device that can measure vital signs based on a determined posture while easily determining body posture.

### BACKGROUND ART

Recently, information and communication technology has developed dramatically, and social awareness of healthcare has significantly changed. Therefore, efforts are being made to establish a medical system centered on prevention rather than treatment-centered medical care, and health management-centered medical systems rather than disease management-centered medical care through the combination of information and communication technology and healthcare technology.

In particular, as electronic devices apply information and communication technology such as wearable watches, the electronice devices may provide body data such as body temperature, pulse, blood oxygen pressure, and electrocardiogram. As a result, vital signs that can confirm changes in user's body can be detected as body data accumulates.

However, when the electronic device measures body data, the reliability of the body data may become problematic as the data varies depending on measurement conditions. For example, an ECG test should be carried out under the condtion that “the patient should lie in a supine position (or semi-sitting position), remain still during the test, and should remove metal from their body in advance [Source: Cardiology ECG Measurement Manual].” When there is no metal on the patient's body, for medically meaningful ECG measurements, firstly, the patient should keep his position to be supine, semi-sitting, or semi-Fowler's position, Secondly, it should be confirmed that the patient is under the sufficiently stable position. Therefore, in a medical test environment, body data needs to be acquired with the patient's position specified.

In the medical test environment, patient postures are largely classified into twelve types: supine position, prone position, lateral position, dorsal recumbent position, lithotomy position, knee-chest position, Sims' position, Fowler's position, semi-Fowler's position, Trendelenburg position, reverse Trendelenburg position, and jackknife position.

Specifically, hospitalized patients' positions are maintained close to basic anatomical positions for comfort, with joints slightly flexed to prevent increased muscle tension and fatigue, and changes in position comply with the normal range of motion (ROM) of joints. For example, in case of patients at risk of pressure ulcers, their positions should be changed every two hours.

All stages of position selection, change, maintenance, and recording are performed solely by medical personnel, and there is no method to automatically determine these conditions.

For example, in hospitals, the four vital signs include respiration, pulse, blood pressure, and body temperature. The four vital signs are all basically measured in the supine position, and this is so fundamental that unless specifically noted otherwise in the medical chart, measurements are assumed to have been taken in the supine position.

The supine position has been the basic nursing position for hundreds of years due to its wide range of benefits, including reduced risk of choking, decreased risk of esophageal reflux, reduced burden of pulmonary function, and increased time of sleep. However, there are cases where specific positions need to be maintained during the post-operative recovery period.

Alternatively, in cases of brain surgery for subdural or subarachnoid hemorrhage, the prone position must be maintained for at least fifteen days. After procedures such as transarterial chemoembolization for cancer treatment, it is known that maintaining a supine position for 24 hours, rather than moving immediately, shows better therapeutic effects.

Furthermore, for patients at risk of developing pressure ulcers, positions are changed regularly. Changing positions at regular intervals between supine, semi-sitting, prone, and lateral positions is an important method for preventing pressure ulcers, which can be a cause of death.

The prone position is frequently used in spine and neck surgery, neurosurgery, colorectal surgery, vascular surgery, and tendon repair. The sitting position is used in neurosurgery and shoulder surgery. The Fowler's position, similar to sitting in a beach chair, is used in nasal surgery, abdominoplasty, and breast surgery.

While such specific positions exist for various nursing and surgical procedures in hospitals, position verification is currently performed only through visual inspection by medical staff. There is a need to obtain time-series vital sign data according to position maintenance conditions.

Meanwhile, wearable digital healthcare devices primarily measure vital signs. The four main vital signs include blood pressure, pulse, respiration, and body temperature. For example, normal blood pressure is 120/80 mmHg systolic/diastolic, normal pulse is 60-100 beats per minute, normal respiration is 12-20 breaths per minute, and normal body temperature ranges from 36-37° C.

These reference values are established under the condition that the subject is in a stable state. Blood pressure immediately after waking is highest during the day as the body is in the process of awakening. During exercise, blood pressure increases to support muscle use by increasing blood circulation speed, but after exercise, systolic blood pressure typically decreases by 5 to 8 mmHg due to exercise effects.

As blood pressure, pulse, respiration, and temperature values all vary simply based on exercise status and intensity, these measurements cannot be used as a basis for determining whether the subject's vital signs are normal. Furthermore, even if vital signs are stable, if it cannot be determined whether the subject was stabilized in a standing or lying position, the measurements lose their utility as medical data.

Additionally, smartwatches can measure blood oxygen saturation and pulse. In this case, data measured while the subject swings his arms may not be used at all for medical purposes. During sleep, these measurement primaily serves to determine sleep status based on reduced movement.

Smartwatch developers claim there is a close correlation between their measurement data using the smartwatch and existing medical device measurement data and argue they can be used as medical data. However, the medical field, which must be conservative due to handling human lives, currently only trusts data measured at hospitals where patients visit.

The conditions for measuring ECG are “have the patient lie in a supine position (or semi-sitting position), ensure they remain still during the examination, and remove any metal from the patient's body [Source: Cardiology ECG Measurement Manual].”

Measurement conditions for other vital signs are similar. Therefore, if it can be confirmed that the subject is stable in a supine or semi-sitting position, subsequent measurements can be used for medical purposes.

Consequently, there is a demand for a vital signs measuring device that can measure vital signs such as pulse rate, blood oxygen pressure, respiratory rate based on SpO2 sensors, Lead I and V1-V6 ECG based on ECG sensors, and blood glucose based on strip sensors, while recognizing that stable positions such as supine or semi-sitting positions have been maintained for a certain period.

Furthermore, since each user may have different primary vital signs they wish to measure, a system capable of measuring everything including pulse/blood oxygen pressure/respiration/ECG I/ECG (V1-V6)/blood glucose necessarily has disadvantages such as high cost, short battery life, and considerable sensor system weight during use. Therefore, there is a demand for a vital signs measuring device that can use only one or two sensor systems as needed and allow additional sensor systems to be added later as required.

### DETAILED DESCRIPTION OF THE INVENTION

### Technical Problem

Embodiments of the present invention provide a vital signs measuring device capable of measuring vital signs in a state where a stable posture has been maintained for a certain period.

### Technical Solution

According to example embodiments of the present invention, a vital signs measuring device includes a posture measuring unit attachable within a pentagon-cuboid-superimposed space of a body, a biosignal measuring unit detachably connected to the posture measuring unit and capable of measuring at least one of the body's vital signs, a wireless communication unit that transmits data calculated from the biosignal measuring unit externally when the posture measuring unit determines the body to be in a specific posture, and a power supply unit that supplies power to the posture measuring unit, biosignal measuring unit and wireless communication unit, wherein the pentagon-cuboid-superimposed space is defined as an interior of a pentagon connecting the left and right pectoralis major apexes, both shoulder deltoid apexes, and the chin.

In an example embodiment, the posture measuring unit includes a sensor that calculates acceleration or angular velocity for each of three absolute axis directions defined where the direction opposite to gravity is the Z-axis direction, the frontal view direction in the body's standing state is the X-axis direction, and the Y-axis direction is defined as the direction generating the Z-axis through vector product of the X-axis direction, a calculator that calculates the sensor's tilt values for each of the three absolute axis directions using the acceleration or angular velocity, a calibrator that derives corrected sensor tilt values by correcting the sensor's tilt values using an own tilt value of the sensor that changes according to the sensor's attachment position when the body is standing, and a determinator that specifies the body's posture from the corrected sensor tilt values.

Here, the sensor comprises an accelerometer or gyro sensor.

Further, when the sensor is an accelerometer, the acceleration value in the X-axis direction is defined as αₓ, the acceleration value in the Z-axis direction is defined as α_{z},
and the acceleration measurement values for each of the X-axis to Z-axis directions are defined as vector \vec{R},
and the acceleration value α_{y} in the Y-axis direction satisfies Mathematical Formulas 1 and 2,
and the sensor's tilt values are defined by Mathematical Formula 3:

$$9.8 = {\sqrt{\vert \vec{{a}_{X}} + \vec{{α}_{Y}} + \vec{{α}_{Z}} \vert}}^{2} \qquad Mathematical Formula 1$$

$$∠ ( \vec{{a}_{X}} + \vec{{α}_{Y}} + \vec{{α}_{Z}} ) = - ∠ \vec{z} \qquad Mathematical Formula 2$$

$$θ xr = arccos ( Rx / R ) , θ Yr = arccos ( RY / R ) , θ zr = arccos ( Rz / R ) \qquad Mathematical Formula 3$$

where θₓᵣ, θ_{Yr} and θ_{zr} are the sensor tilts in the X-axis, Y-axis and Z-axis directions, respectively, Rₓ, R^{Y} and R_{z} are the magnitudes of \vec{R} in each direction, and R is the magnitude of vector \vec{R}.

Furthermore, the calibrator is configured to correct the sensor's tilt values by aligning the three local axis directions defined by the local coordinate system according to the three absolute axis directions.

Here, wherein the calibrator is configured to correct the sensor's tilt values using Mathematical Formula 4:

$$[ \begin{matrix}{θ}_{Xr} \\ {θ}_{Yr} \\ {θ}_{Zr}\end{matrix} ] \cdot [ \begin{matrix}cos β & 0 & sin β \\ 0 & 1 & 0 \\ - sin β & 0 & cos β\end{matrix} ] = [ \begin{matrix}{θ}_{Xr}^{′} \\ {θ}_{Yr}^{′} \\ {θ}_{Zr}^{′}\end{matrix} ] \qquad Mathematical Formula 4$$

where θ_{Xr}, θ_{Yr}, and θ_{Zr} are the sensor inclination vlaues for the X-axis, Y-axis, and Z-axis directions, respectively,
θ_{Xr}′, θ_{Yr}′, and θ_{Zr}′ are the corrected sensor inclinations values for the X-axis, Y-axis, and Z-axis directions, respectively,
and β is a rotation angle required to rotate the z-axis direction of sensor's local coordinate system counterclockwise to align the z-axis direction of sensor's local coordinate system with the Z-axis direction of the absolute coordinate system when the patient is standing.

In an example embodiment, the biosignal measuring unit comprises at least one of a PPG sensor, temperature sensor, standard ECG lead sensor, and blood glucose sensor.

In an example embodiment, the posture measuring unit, biosignal measuring unit, and wireless communication unit transmit and receive data using I2C communication method.

Here, the posture measuring unit functions as master mode and the biosignal measuring unit functions as slave mode.

### Advantageous Effects

According to the present invention, vital signs can be measured in a state where a stable posture has been maintained for a certain period. Only one to two sensor modules can be used as needed and sensor modules can be added later as needed. Further, reliable vital signs data can be obtained for specific body postures.

### BRIEF DESCRIPTION OF THE DRAWINGS

FIG. 1 is a photograph defining the X-axis, Y-axis, and Z-axis directions and the pentagon-cuboid-superimposed space based on patient's standing state;

FIG. 2 is a block diagram illustrating a vital signs measuring device with accordance to an example embodiment of the present invention;

FIG. 3 is a plan view illustrating the vital signs measuring device of FIG. 2;

FIG. 4 is a block diagram illustrating I2C communication of the communication unit of FIG. 2;

FIG. 5 is a flowchart illustrating a method for determining body posture using the posture measuring unit of FIG. 2;

FIG. 6 is a schematic diagram illustrating an example of determining a semi-sitting position;

FIG. 7 is an azimuthal coordinate system for explaining the method of calculating sensor tilt values using acceleration measurements;

FIG. 8 is a schematic diagram illustrating the differences between the defined directions of the X-axis, Y-axis, and Z-axis directions defined in FIG. 1 and the own directions of the sensor;

FIG. 9 is a schematic diagram illustrating the step of correcting sensor tilt values using the sensor's inherent tilt values; and

FIG. 10 is a flowchart illustrating the operation of a vital signs measuring device according to embodiments of the present invention.

### BEST MODE FOR CARRYING OUT THE INVENTION

Hereinafter, embodiments of the present invention will be described in detail with reference to the accompanying drawings. Since the present invention may be variously modified and have various forms, specific embodiments will be illustrated in the drawings and described in detail in the text. However, it should be understood that this is not intended to limit the present invention to the specific disclosed forms, and includes all modifications, equivalents, and substitutes included in the spirit and technical scope of the present invention. In describing each drawing, similar reference numerals are used for similar components. In the accompanying drawings, the dimensions of the structures are shown enlarged than actual size for clarity of the present invention.

Although terms such as first, second, etc. may be used to describe various components, the components should not be limited by these terms. These terms are only used to distinguish one component from another component. For example, a first component could be termed a second component, and similarly, a second component could be termed a first component, without departing from the scope of the present invention.

The terminology used herein is for the purpose of describing particular embodiments only and is not intended to limit the present invention. The singular forms are intended to include the plural forms as well, unless the context clearly indicates otherwise. In this application, it should be understood that terms such as “include” or “have” are intended to designate the presence of features, numbers, steps, operations, components, parts, or combinations thereof described in the specification, but do not preclude the possibility of the presence or addition of one or more other features, numbers, steps, operations, components, parts, or combinations thereof.

Unless otherwise defined, all terms used herein, including technical or scientific terms, have the same meaning as commonly understood by one of ordinary skill in the art to which the present invention belongs. Terms such as those defined in commonly used dictionaries should be interpreted as having a meaning that is consistent with their meaning in the context of the relevant art, and will not be interpreted in an idealized or overly formal sense unless expressly so defined in this application.

### MODE FOR THE INVENTION

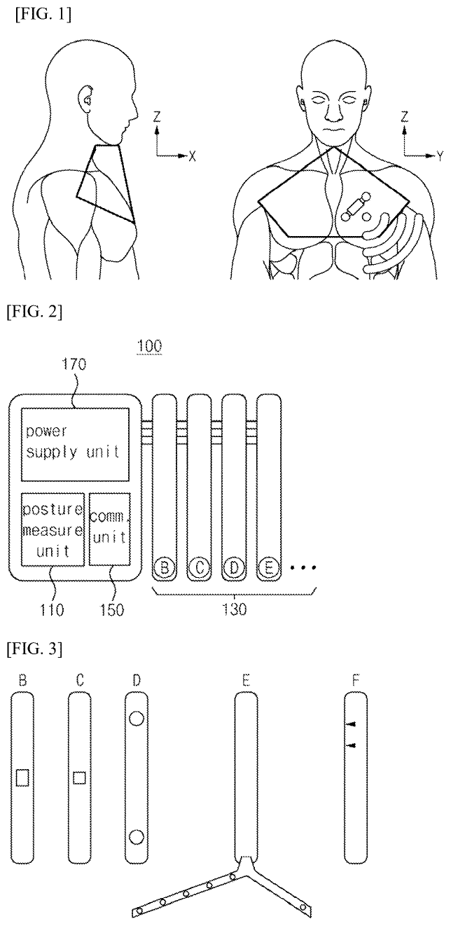

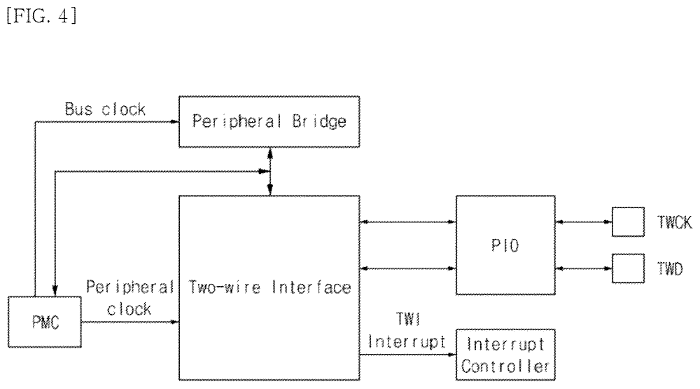

FIG. 1 is a photograph defining the X-axis, Y-axis, and Z-axis directions and the pentagon-cuboid-superimposed space based on a patient's standing state. FIG. 2 is a block diagram illustrating a vital signs measuring device according to an example embodiment of the present invention. FIG. 3 is a plan view illustrating the vital signs measuring device of FIG. 2. FIG. 4 is a block diagram illustrating I2C communication of the wireless communication unit of FIG. 2.

Referring to FIGS. 1 to 4, a vital signs measuring device 10 according to an example embodiment of the present invention includes a posture measuring unit 110, a biosignal measuring unit 130, a wireless communication unit 150, and a power supply unit 170.

The posture measuring unit 110 can be attached within a pentagon-cuboid-superimposed space of a body. The configuration and method by which the posture measuring unit 110 determines the body's posture will be described later.

The biosignal measuring unit 130 is configured to be connectable to the posture measuring unit 110. Specifically, the biosignal measuring unit 130 can be connected to the posture measuring unit 110 with including multiple modularized measurement modules. Therefore, the biosignal measuring unit 130 can use only one or two sensor modules as needed and additional sensor modules can be added later as required.

The biosignal measuring unit 130 can measure at least one of body's vital signs. For example, the biosignal measuring unit 130 can measure pulse rate, blood oxygen pressure, respiratory rate based on blood oxygen saturation (SpO2) sensors, Lead I and V1-V6 ECG based on ECG sensors, and blood glucose based on strip sensors.

Referring to FIG. 3, B indicates a PPG sensor, C indicates a temperature sensor, D indicates a standard ECG Lead I sensor, E indicates ECG V1-V6 lead sensors, and F indicates a blood glucose sensor.

Meanwhile, the wireless communication unit 150 transmits data calculated by the posture measuring unit 110 and biosignal measuring unit 130 externally. The wireless communication unit 150 performs data transmission using an I2C communication method.

Referring again to FIGS. 3 and 4, the I2C (Inter-integrated circuit) communication method uses lines which includes an SDA line for exchanging data and an SCL line corresponding to a clock line for synchronizing timing.

At this time, the I2C communication method consists of one master mode and another slave mode. And at maximum, 127 slaves can be provided.

Here, each measurement module (B, C, D, E . . . ) included in the biosignal measuring unit 130 includes SDA and SCL terminals required for the I2C communication method. All measurement modules share a total of four terminals including SDA terminal, SCL terminal, and power terminals −(ground) and +(power) connected to the power supply unit 170.

Meanwhile, the posture measuring unit 110 functions as the master mode for communication, while the remaining measurement modules operate in slave mode. Each slave is assigned to have an address between 0 and 127 and transmits measurement data to the posture measuring unit through SDA and SCL lines at the master's request.

Subsequently, the posture measuring unit 110 outputs data externally through the wireless communication unit 150. When the addresses of the wireless communication unit 150 for the target vital signs and slaves are fixed, the biosignal measuring unit 130 can send data to the assigned address according to master's commands provided to its slave address, regardless of the order in which it is coupled to the posture measuring unit 110.

Therefore, when the biosignal measuring unit 130 includes measurement modules B through F, addresses can be assigned as B: 0x01, C: 0x02, D: 0xA, E: 0x0C, F: 0xOF. Thus, the posture measuring unit 110 can output measurement data read from each measurement module externally through the wireless communication unit 150 regardless of the coupling order. In this way, the biosignal measuring unit 130 can add or subtract up to 127 measurement modules.

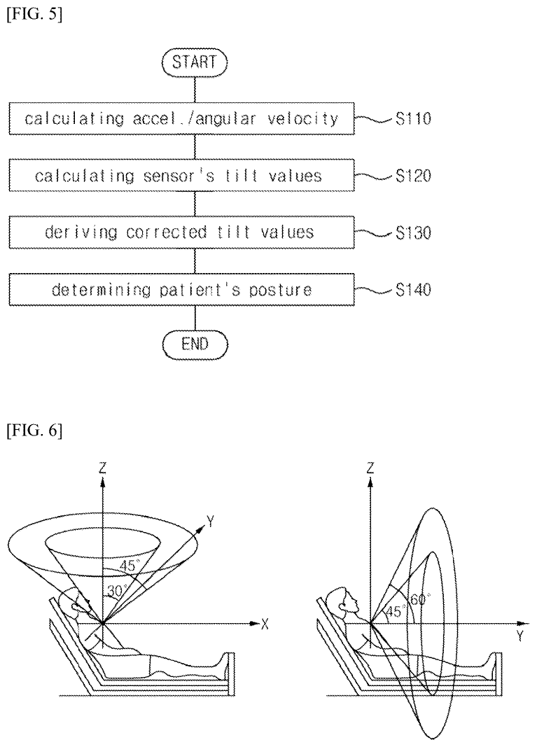

FIG. 5 is a flowchart illustrating a method for determining body posture. FIG. 6 is a schematic diagram illustrating an example of determining a semi-sitting position;

Referring to FIGS. 5 and 6, the posture measuring unit 110 according to example embodiments of the present invention includes a sensor, calculator, calibrator, and determinator.

Directions according to an absolute coordinate system are defined such that the direction opposite to gravity is the Z-axis direction, the frontal view direction in the body's standing state is the X-axis direction, and the Y-axis direction is defined as the direction generating the Z-axis through vector product of the X-axis direction. This defines each of the three axis directions according to the absolute coordinate system.

Meanwhile, regarding the local coordinate system of the sensor itself, three axis directions including x-axis, y-axis, and z-axis directions are defined in a local coordinate system. The local coordinate system will be described later with reference to FIG. 8.

Firstly, the sensor may measure acceleration or angular velocity for each of the three axis directions according to the absolute coordinate system.

The calculator calculates sensor's tilt values for each of the three axis directions using the acceleration or angular velocity.

The calibrator derives a corrected sensor tilt values by correcting sensor's tilt values using an own tilt value of the sensor that may change according to the sensor's attachment position when the body is standing.

The determinator specifies the patient's posture from the corrected sensor tilt values. At this time, the pentagon-cuboid-superimposed space is defined as the interior of a pentagon connecting the patient's left and right pectoralis major apexes, both shoulder deltoid apexes, and chin.

The operation of the posture measuring unit will now be described.

First, using a sensor attached within the patient's pentagon-cuboid-superimposed space, acceleration or angular velocity is calculated for each of the three axis directions according to the absolute coordinate system (S110).

At this time, the pentagon-cuboid-superimposed space is defined as the interior of a pentagon connecting the patient's left and right pectoralis major apexes, both shoulder deltoid apexes, and chin. In particular, the sensor can be attached to the patient's anterior mediastinum.

Thus, the sensor does not cause discomfort to the user by being pressed even in the patient's lying position. Additionally, the sensor facilitates discrimination of supine, semi-sitting (Fowler's position), and semi-Fowler's posture which are frequently applied during medical diagnosis. Furthermore, it becomes possible to determine whether the lateral position that can reduce the burden of sleep apnea is right lateral or left lateral.

Moreover, when the sensor is positioned in the pentagon-cuboid-superimposed space, it can obtain the most accurate acceleration or angular velocity values.

The sensor includes, for example, an accelerometer or gyro sensor. In the case of the accelerometer, acceleration is measured, and in the case of the gyro sensor, angular velocity can be measured.

Patient postures are broadly classified into twelve types and can be categorized as supine position, prone position, lateral position, dorsal recumbent position, lithotomy position, knee-chest position, Sims' position, Fowler's position, semi-Fowler's position, Trendelenburg position, reverse Trendelenburg position, and jackknife position.

Regarding the patient's posture, the examination areas and application situations can be summarized as shown in Table 1 below.

TABLE 1

| Position | Examination Area | Application Situation |
|---|---|---|
| Supine Position | Axilla, heart, abdomen, pulse | Male catheterization, spinal fracture, before |
| | measurement | consciousness recovery after surgery, ECG |
| | | measurement |
| Semi-sitting | — | Dyspnea, consciousness recovery after surgery |
| Position | | |
| Knee-chest Position | Rectum, rectoscopy | Fetal position correction, menstrual pain relief, |
| | | prevention of uterine retroversion, fetal |
| | | survival after umbilical cord prolapse |
| Lithotomy Position | Female reproductive organs, | Delivery |
| | cervical cancer examination, | |
| | cystoscopy | |
| Dorsal Recumbent | Abdominal examination | Female catheterization, abdominal hernia |
| Position | (pressure removal) | |
| Sims' Position | Rectum, vagina | Endoscopic examination |
| (lateral) | | |
| Prone Position | Pelvic joint extension | Post-diaphragmatic hemorrhage drainage |
| | | surgery, back massage |

Next, the sensor's tilt values for each of the three axis directions according to the absolute coordinate system are calculated using the acceleration or angular velocity (S120).

At this time, an acceleration value ay in the Y-axis direction satisfies Mathematical Formulas 1 and 2 below:

$$9.8 = {\sqrt{\vert \vec{{a}_{X}} + \vec{{α}_{Y}} + \vec{{α}_{Z}} \vert}}^{2} \qquad Mathematical Formula 1$$

$$∠ ( \vec{{a}_{X}} + \vec{{α}_{Y}} + \vec{{α}_{Z}} ) = - ∠ \vec{Z} \qquad Mathematical Formula 2$$

That is, the sensor receives gravitational acceleration of 9.8 m/s² in the direction toward the earth's center, i.e., the gravity direction. This is because if the sensor is attached to the chest of a standing patient and the patient with the attached sensor is in a standing position, acceleration of 9.8 m/s² is acting in the negative direction of the z-axis, which is opposite to the gravity direction.

Therefore, when using an accelerometer, in a state without patient fluctuation, the vector sum of the sensor's acceleration values always has a magnitude of 1 G (9.8 m/sec²) and is a vector in the (−) Z direction. In other words, the magnitude of the vector sum of each of the X, Y and Z axis accelaration values of the stationary accelerometer (square root of the sum of squares of the three values) always converges to 9.8.

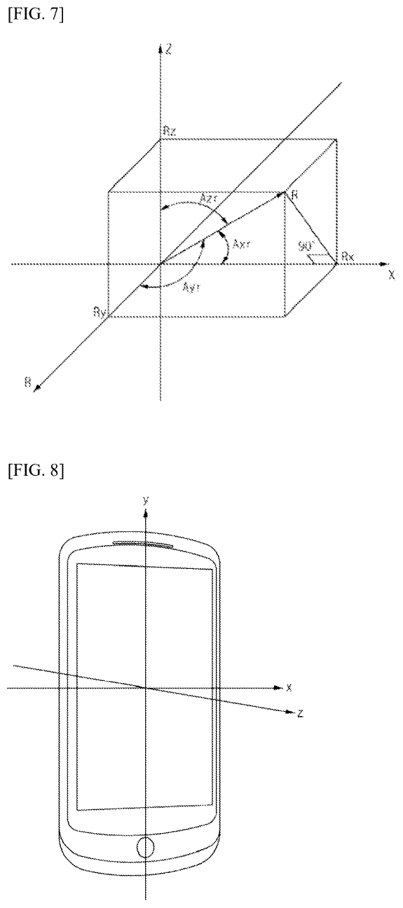

FIG. 7 is an azimuthal coordinate system for explaining the method of calculating sensor tilt values using acceleration measurements.

Referring to FIG. 7, the acceleration values for each of the X-axis to Z-axis directions are defined as vector \vec{R}, and the sensor's tilt values are defined by Mathematical Formula 3:

$$θ xr = arccos ( Rx / R ) , θ Yr = arccos ( RY / R ) , θ zr = arccos ( Rz / R ) \qquad Mathematical Formula 3$$

where θₓᵣ, θ_{Yr} and θ_{zr} are the sensor tilts in the X-axis, Y-axis and Z-axis directions, respectively, Rₓ, R^{Y} and R_{z} are the magnitudes of \vec{R} in each direction, and R is the magnitude of vector \vec{R}.

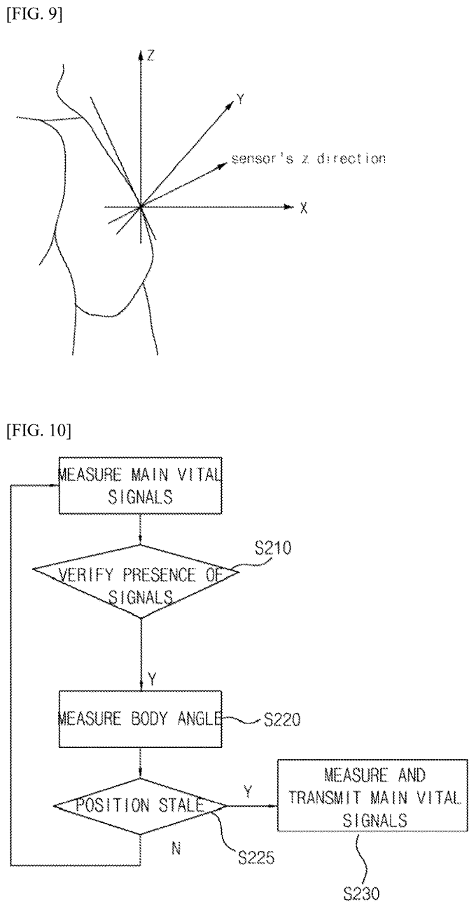

FIG. 8 is a schematic diagram illustrating the differences between the defined directions of the X-axis, Y-axis, and Z-axis in FIG. 1 and the own directions of the sensor. FIG. 9 is a schematic diagram illustrating the step of correcting sensor tilt values using the sensor's inherent tilt values.

Referring to FIGS. 1, 8, and 9, corrected sensor tilt values are derived by correcting the sensor's tilt values using an own tilt value of the sensor that changes in a local coordinate system according to the sensor's attachment position (S130).

That is, the sensor's gravity value may vary depending on an attachment position, which is part of the patient's body. Therefore, correction sensor's tilt values using an own tilt value of the sensor is necessary.

More specifically, to correct the sensor's tilt values using the sensor's inherent tilt value when the patient is standing, the sensor's own z-axis direction, which varies according to the sensor's attachment position, can be aligned with the Z-axis direction according to the absolute coordinate system.

For example, a body angle measurement sensor attached to the patient's anterior mediastinum has its circuit board chip-attached surface as the x-y plane, corresponding to the attachment surface of the anterior mediastinum, and the normal direction extended through the chip is defined as the sensor's own z-axis direction. Therefore, the local coordinate system including the sensor's own x-axis, y-axis, and z-axis directions attached to the patient's pentagon-cuboid-superimposed space differs from the absolute coordinate system based on the space where the vertical direction in the patient's standing state is the Z-axis.

In this case, the step of aligning the own z-axis direction of the sensor with the Z-axis direction includes rotation of the own z-axis direction by 60 to 70 degrees. This is because the patient's chest is inclined at 20 to 30 degrees relative to the gravity direction, (−) Z-axis direction.

Specifically, corrected sensor inclination values (θ_{Xr}′, θ_{Yr}′, and θ_{Zr}′) are derived through matrix transformation using Mathematical Formula 4 below;

$$[ \begin{matrix}{θ}_{Xr} \\ {θ}_{Yr} \\ {θ}_{Zr}\end{matrix} ] \cdot [ \begin{matrix}cos β & 0 & sin β \\ 0 & 1 & 0 \\ - sin β & 0 & cos β\end{matrix} ] = [ \begin{matrix}{θ}_{Xr}^{′} \\ {θ}_{Yr}^{′} \\ {θ}_{Zr}^{′}\end{matrix} ] \qquad Mathematical Formula 4$$

Here, θ_{Xr}, θ_{Yr}, and θ_{Zr} are the sensor inclination vlaues for the X-axis, Y-axis, and Z-axis directions, respectively,
θ_{Xr}′, θ_{Yr}′, and θ_{Zr}′ are the corrected tilt values for the X-axis, Y-axis, and Z-axis directions, respectively,
and β is a rotation angle required to rotate the z-axis direction of the local coordinate system counterclockwise to align the z-axis direction of the local coordinate system with the Z-axis direction of the absolute coordinate system when the patient is standing.

Subsequently, the patient's posture is determined from the corrected sensor tilt values (S140).

The corrected sensor tilt values θ_{Xr}′, θ_{Yr}′ and θ_{Zr}′ correspond to the corrected sensor tilts in the X-axis, Y-axis and Z-axis directions, respectively.

TABLE 2

| | X-axis | Y-axis | Z-axis |
|---|---|---|---|
| | Inclination | Inclination | Inclination |
| Position | Angle (θ_{X}r′, °) | Angle (θ_{Y}r′, °) | Angle (θ_{Z}r′, °) |
| Supine Position | 0 | 0 | 90(−90) |
| Semi-sitting Position | 45~60 | 0 | 30~45 |
| (Fowler's) | | | |
| Semi-Fowler's | 25~30 | 0 | 60~65 |
| Position | | | |
| Lateral Position | 90(−90) | 0 | 90(−90) |
| Prone Position | −180 | 90(−90) | 90(−90) |
| Trendelenburg | −10 | 0 | >100(−100) |
| Position | | | |

FIG. 10 is a flowchart illustrating a method for acquiring vital signs data from a nursing patient according to embodiments of the present invention.

Referring to FIG. 10, in a method for acquiring vital signs data from a nursing patient according to embodiments of the present invention, the presence of signals related to the patient's preliminary vital signs is first verified (S210). The preliminary vital signs, which is unique to the human body, includes body temperature, respiration, pulse, blood oxygen saturation, and ECG, for example. The preliminary vital signs are measured first and processed as start commands.

Subsequently, if a signal processed as a start command exists, the patient's posture is determined by measuring body angles (S220). The eligibility of the patient's posture is determined (S225). Afterward, the patient's main vital signs are measured, and the data related to the main vital signs is transmitted (S230).

To determine the patient's position, a sensor attached within the patient's pentagon-cuboid-superimposed space is used, where the gravity direction is defined as the Z-axis direction, and the front view direction in the patient's standing position is defined as the X-axis direction, to calculate acceleration or angular velocity for each of the three axis directions. Then, using this acceleration or angular velocity, the sensor's inclination values for each of the three axis directions are calculated. Corrected sensor inclination values are derived by correcting the sensor inclination values using the sensor's own inclination value based on its attachment position. Subsequently, the patient's position is determined from these corrected sensor inclination values. Here, the pentagon-cuboid-superimposed space is defined as the interior of a pentagon connecting the patient's left and right pectoralis major apices, both shoulder deltoid apices, and chin.

The device according to embodiments of the present invention may include a processor that performs each step constituting the body position determination method by processing data, a memory that stores program data, a permanent storage such as a disk drive, a communication port that communicates with external devices and user interface devices such as a touch panel, keys, buttons, etc. Methods implemented as software modules or algorithms can be stored as computer-readable codes or program instructions executable on the processor on a computer-readable recording medium. Computer-readable recording media may include magnetic storage media (e.g., ROM (read-only memory), RAM (random-access memory), floppy disks, hard disks, etc.), optical reading media (e.g., CD-ROMs, DVDs (Digital Versatile Discs)), and the computer-readable recording medium can be distributed among computer systems connected through a network, and computer-readable codes can be stored and executed in a distributed manner. The media is computer-readable, can be stored in memory, and can be executed by a processor.

The embodiments of the present invention can be represented by functional blocks and various processing steps. These functional blocks can be implemented by various numbers of hardware and/or software configurations that execute specific functions. For example, the embodiment may employ integrated circuit configurations such as a memory, a processor, a logic device, look-up tables, etc. which can execute various functions under the control of one or more microprocessors or other control devices. Similar to how components of the invention can be executed by software programming or software elements, embodiments can be implemented by various algorithms including combinations of data structures, processor, routines, other programming configurations implemented in programming or scripting languages such as C, C++, Java, assembler, etc. Functional aspects can be implemented as algorithms executed on one or more processors. Also, the embodiment may employ conventional technology for electronic configuration, signal processing, and/or data processing. Terms such as “mechanism”, “element”, “means”, “configuration” can be used broadly and are not limited to mechanical and physical configurations. These terms may include the meaning of a series of software routines in association with a processor, etc.

### INDUSTRIAL APPLICABILITY

The particular implementations described in this specification are examples and do not in any way limit the scope of the embodiment. For the sake of brevity in the specification, descriptions of conventional electronic configurations, control systems, software, and other functional aspects of the systems may be omitted. Furthermore, the connecting lines or connecting members between components shown in the drawings are intended to illustrate functional connections and/or physical or circuit connections by way of example and may be represented in actual devices by alternative or additional functional connections, physical connections, or circuit connections.

Finally, the specific implementations described in this specification are examples of implementing the technical idea of the present invention and various modifications are possible within the scope of the technical idea of the present invention. Therefore, although specific embodiments have been shown and described herein, various modifications and changes may be made without departing from the disclosed embodiments. The scope of the present invention is not limited by the foregoing description but is defined by the appended claims, and all differences within equivalent scope should be construed as being included in the present invention.

1. A vital signs measuring device comprising:
a posture measuring unit attachable within a pentagon-cuboid-superimposed space of a body;
a biosignal measuring unit detachably connected to the posture measuring unit and capable of measuring at least one of the body's vital signs;
a wireless communication unit that transmits data calculated from the biosignal measuring unit externally when the posture measuring unit determines the body to be in a specific posture; and
a power supply unit that supplies power to the posture measuring unit, biosignal measuring unit and wireless communication unit,
wherein the pentagon-cuboid-superimposed space is defined as an interior of a pentagon connecting the left and right pectoralis major apexes, both shoulder deltoid apexes, and the chin.

2. The vital signs measuring device of claim 1, wherein the posture measuring unit comprises:
a sensor that calculates acceleration or angular velocity for each of three absolute axis directions defined where the direction opposite to gravity is the Z-axis direction, the frontal view direction in the body's standing state is the X-axis direction, and the Y-axis direction is defined as the direction generating the Z-axis through vector product of the X-axis direction;
a calculator that calculates the sensor's tilt values for each of the three absolute axis directions using the acceleration or angular velocity;
a calibrator that derives corrected sensor tilt values by correcting the sensor's tilt values using an own tilt value of the sensor that changes according to the sensor's attachment position when the body is standing; and
a determinator that specifies the body's posture from the corrected sensor tilt values.

3. The vital signs measuring device of claim 2, wherein the sensor comprises an accelerometer or gyro sensor.

4. The vital signs measuring device of claim 2, wherein when the sensor is an accelerometer, the acceleration value in the X-axis direction is defined as αₓ, the acceleration value in the Z-axis direction is defined as α_{z}, and the acceleration measurement values for each of the X-axis to Z-axis directions are defined as vector \vec{R}, and the acceleration value α_{y} in the Y-axis direction satisfies Mathematical Formulas 1 and 2, and the sensor's tilt values are defined by Mathematical Formula 3:
$$9.8 = {\sqrt{\vert \vec{{a}_{X}} + \vec{{α}_{Y}} + \vec{{α}_{Z}} \vert}}^{2} \qquad Mathematical Formula 1$$
$$∠ ( \vec{{a}_{X}} + \vec{{α}_{Y}} + \vec{{α}_{Z}} ) = - ∠ \vec{z} \qquad Mathematical Formula 2$$
$$θ xr = arccos ( Rx / R ) , θ Yr = arccos ( RY / R ) , θ zr = arccos ( Rz / R ) \qquad Mathematical Formula 3$$
where θ_{Xr}, θ_{Yr} and θ_{Zr} are the sensor tilts in the X-axis, Y-axis and Z-axis directions, respectively, Rₓ, R^{Y} and R_{z} are the magnitudes of \vec{R} in each direction, and R is the magnitude of vector \vec{R}.

5. The vital signs measuring device of claim 2, wherein the calibrator is configured to correct the sensor's tilt values by aligning the three local axis directions defined by the local coordinate system according to the three absolute axis directions.

6. The vital signs measuring device of claim 2, wherein the calibrator is configured to correct the sensor's tilt values using Mathematical Formula 4:
$$[ \begin{matrix}{θ}_{Xr} \\ {θ}_{Yr} \\ {θ}_{Zr}\end{matrix} ] \cdot [ \begin{matrix}cos β & 0 & sin β \\ 0 & 1 & 0 \\ - sin β & 0 & cos β\end{matrix} ] = [ \begin{matrix}{θ}_{Xr}^{′} \\ {θ}_{Yr}^{′} \\ {θ}_{Zr}^{′}\end{matrix} ] \qquad Mathematical Formula 4$$
where:
θ_{Xr}, θ_{Yr}, and θ_{Zr} are the sensor inclination vlaues for the X-axis, Y-axis, and Z-axis directions, respectively,
θ_{Xr}′, θ_{Yr}′, and θ_{Zr}′ are the corrected sensor inclinations values for the X-axis, Y-axis, and Z-axis directions, respectively, and
β is a rotation angle required to rotate the z-axis direction of sensor's local coordinate system counterclockwise to align the z-axis direction of sensor's local coordinate system with the Z-axis direction of the absolute coordinate system when the patient is standing.

7. The vital signs measuring device of claim 1, wherein the biosignal measuring unit comprises at least one of a PPG sensor, temperature sensor, standard ECG lead sensor, and blood glucose sensor.

8. The vital signs measuring device of claim 1, wherein the posture measuring unit, biosignal measuring unit, and wireless communication unit transmit and receive data using I2C communication method.

9. The vital signs measuring device of claim 8, wherein the posture measuring unit functions as master mode and the biosignal measuring unit functions as slave mode.
